(19) United States
(12) Patent Application Publication
(10) Pub. No.: US 2026/0276564 A1
(43) Pub. Date: Sep. 17, 2026

(54) HIGH-SPEED CT SCANNER SYSTEM WITH X-RAY TUBE PRODUCING LINEAR X-RAY SOURCES

(71) Applicant: MEDICHORD LLC, Lewes, DE (US)

(72) Inventors: YU ZOU, Lewes, DE (US); Yi YANG, Lewes, DE (US); Daxin SHI, Lewes, DE (US)

(21) Appl. No.: 19/677,936

(22) Filed: May 14, 2026

Related U.S. Application Data
(63) Continuation of application No. PCT/US2024/056938, filed on Nov. 21, 2024.
(60) Provisional application No. 63/649,861, filed on May 20, 2024.

(51) Int. Cl. G01N 23/046; H05G 1/60

(52) U.S. Cl. CPC G01N 23/046; H05G 1/60; G01N 2223/1016; G01N 2223/102; G01N 2223/204; G01N 2223/313; G01N 2223/316; G01N 2223/3303; G01N 2223/501

(57) ABSTRACT

This application provides the structure of an x-ray tube that produces a sequence of linear x-ray sources. An x-ray tube includes a rotatable member having a rotation axis and a helical fin rigidly attached to the rotatable member. The helical fin is configured to rotate together with the rotatable member around the rotation axis. The x-ray tube further includes an electron beam generator configured to generate at least one electron beam. The at least one electron beam is directed to bombard the helical fin as the helical fin rotates, producing x-rays whose focal spot moves in space along a line defined by the at least one electron beam.

### RELATED APPLICATION

This application is a continuation of PCT application No. PCT/US2024/056938, entitled “HIGH-SPEED CT SCANNER SYSTEM WITH X-RAY TUBE PRODUCING LINEAR X-RAY SOURCES” filed on Nov. 21, 2024, which claims priority to U.S. Provisional Patent Application No. 63/649,861, entitled “NOVEL IMPLEMENTATION OF AN X-RAY TUBE PRODUCING LINEAR X-RAY SOURCES AND ITS APPLICATION TO BUILD A HIGH-SPEED MEDICAL CT SCANNER” filed on May 20, 2024, the contents of which is incorporated herein by reference in their entirety.

### TECHNICAL FIELD

The disclosed embodiments relate generally to Computed Tomography (CT) scanning, including but not limited to high-speed CT scanners with x-ray tubes producing linear x-ray sources.

### BACKGROUND

X-ray scanning technology is used in many industrial and commercial applications, most notably in security and healthcare. Security analysts and operators utilize x-ray scanners to detect hidden items in baggage, cargo containers, personal and commercial vehicles. Physicians use medical x-ray scanners to assess the health of a patient, locate hidden health risks such as tumorous growths, and for non-intrusive observation of trauma such as broken bones.

Computed tomography is a type of x-ray scanning and is commonly referred to as a CT scan. A CT scan is a diagnostic imaging procedure that uses a combination of X-rays and computer technology to produce images of the inside of the body. During a CT scan, the patient lies on a bed that slowly moves through the gantry while the x-ray tube rotates around the patient, shooting narrow beams of x-rays through the body. The x-ray detectors of the CT scanners are located directly opposite the x-ray source. As the x-rays leave the patient, they are picked up by the detectors. The signals from the detectors are digitized and transmitted to a computer.

In many clinical applications, patient motion is inevitable when taking a CT scan. In some situations, even if the patient stays still during the scan, the portion of the patient's body, such as his or her heart, continues to beat, causing unwanted motions. Such motions cause artifacts such as blurring, streaking, shading, or frame missing. One of the most reliable way to reduce motion artifacts is to make the scan faster which demands the gantry of a CT scanner rotate at a very high speed. However, commercial scanners currently available have their challenges to increase the gantry rotation speed due to the increased G-force exerted on the components (especially the fragile x-ray tube) mounted on the rotating gantry while the gantry rotation speed increases. Accordingly, there is a need for CT scanners capable of providing high-speed imaging without exerting extensive G-force to the x-ray tube.

### SUMMARY

In this application, a novel implementation of an x-ray tube producing linear x-ray sources is proposed, which can be used to build a high-speed medical CT scanner system. By high-speed, it is meant that the gantry rotation speed, if any, can be less than 0.1 sec per revolution. Due to the very limited time allowed for collecting projection data, the high-speed CT scanner will have a great impact on medical imaging problems. The proposed x-ray tubes and associated power electronics will be mounted outside the rotating gantry and remain stationary in the high-speed CT scanner. For example, when the gantry rotates at a very high speed, the CT scanner equipped with the proposed x-ray tubes producing linear x-ray sources can effectively freeze the motion of a human heart, which consequently provides better image quality for cardiac imaging applications. The high-speed CT scanner can also be operated in normal speed mode which is suitable for routine CT applications.

In accordance with some embodiments, an x-ray tube is provided. The x-ray tube comprises a rotatable member having a rotation axis; a helical fin rigidly attached to the rotatable member, wherein the helical fin is configured to rotate together with the rotatable member around the rotation axis; and an electron beam generator configured to generate at least one electron beam. The at least one electron beam is directed to bombard the helical fin as the helical fin rotates, producing x-rays whose focal spot moves in space along a line defined by the at least one electron beam.

In accordance with some embodiments, a CT scanner is provided. The CT scanner comprises a plurality of x-ray tubes, each x-ray tube having a structure according to claim 1, wherein the plurality of x-ray tubes are stationary and form a closed polyline in 3D space around a field of view (FOV) centering around an isocenter; a gantry, wherein the gantry is configured to rotate around the FOV; and a first detector array rigidly attached to the gantry, wherein the first detector array is configured to rotate together with the gantry around the FOV for collecting x-rays generated by the plurality of x-ray tubes. The rotation of the first detector array and the rotation of a helical fin of a corresponding x-ray tube opposite to the first detector array is synchronized such that a real focal spot on the helical fin, a virtual source at a focus of the first detector array, and the isocenter are colinear at every moment.

In accordance with some embodiments, a CT scanner is provided. The CT scanner comprises a plurality of x-ray tubes, each x-ray tube having a structure according to some embodiments of this invention. The plurality of x-ray tubes are stationary and form a closed polyline in 3D space around a field of view (FOV). The CT scanner further comprises a detector array, wherein the detector array is stationary and surrounding the FOV and configured to collect x-rays generated by the plurality of x-ray tubes.

The x-ray tubes and CT scanners described herein may be used not only in the medical imaging field but also in industrial and homeland security applications and many more. The features and advantages described in the specification are not necessarily all inclusive and, in particular, some additional features and advantages will be apparent to one of ordinary skill in the art in view of the drawings, specification, and claims provided in this disclosure. Moreover, it should be noted that the language used in the specification has been principally selected for readability and instructional purposes and has not necessarily been selected to delineate or circumscribe the subject matter described herein.

### BRIEF DESCRIPTION OF THE DRAWINGS

To describe the technical solutions in embodiments of this application more clearly, the following briefly describes the accompanying drawings that need to be used in the description of the embodiments. Apparently, the accompanying drawings described below are merely some exemplary embodiments of this application, and a person of ordinary skill in the art may still derive other accompanying drawings from these accompanying drawings without creative efforts. The following accompanying drawings are not intentionally scaled to an actual size, and emphasis is placed on showing the main idea of this application.

FIG. 1 is a schematic diagram illustrating a conventional CT system.

FIG. 2 is a schematic diagram of a CT scanner in accordance with some embodiments.

FIG. 3 is a schematic diagram of an x-ray tube in accordance with some embodiments.

FIG. 4 is a schematic diagram illustrating one design of an x-ray tube in accordance with some embodiments.

FIG. 5 is a schematic diagram illustrating the design of a fan-angle collimator surrounding an x-ray tube in accordance with some embodiments.

FIG. 6 is a schematic diagram illustrating the design of a cone-angle collimator in accordance with some embodiments.

FIG. 7 is a schematic diagram illustrating a high-speed CT scanner setup including multiple x-ray tubes in accordance with some embodiments.

FIG. 8 is a schematic diagram illustrating a high-speed CT scanner setup including multiple x-ray tubes in accordance with some embodiments.

FIG. 9 is a schematic diagram illustrating a high-speed CT scanner setup with dual detectors in accordance with some embodiments.

FIG. 10 is a schematic diagram illustrating a high-speed CT scanner setup with a stationary detector in accordance with some embodiments.

FIG. 11 is a schematic diagram illustrating a high-speed CT scanner setup with a stationary detector in accordance with some embodiments.

FIG. 12 is a schematic diagram illustrating a high-speed CT scanner setup with densely arranged detector elements in accordance with some embodiments.

FIG. 13 is a schematic diagram illustrating a high-speed CT scanner setup with sparsely arranged detector elements in accordance with some embodiments.

FIG. 14 is a schematic diagram illustrating a high-speed CT scanner setup with a closed polyline scan trajectory in 3D space.

### DETAILED DESCRIPTION

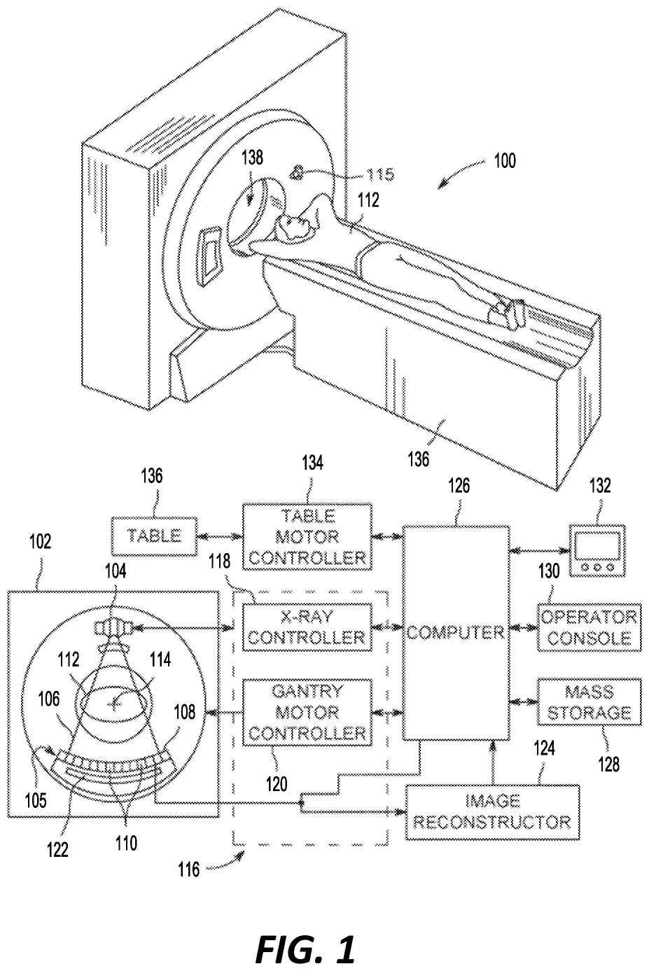

As noted above, conventional CT scanners include a rotating gantry where both the x-ray tubes and the detector are mounted. For example, FIG. 1 is a schematic diagram illustrating a conventional CT system 100 including a rotatable gantry 102. The gantry 102 has an x-ray source 104 that projects a beam of x-rays 106 toward a detector assembly 105 on the opposite side of the gantry 102. The detector assembly 105 includes a collimator assembly 108, a plurality of detector modules 110, and data acquisition systems 122. The plurality of detector modules 110 detect the projected x-rays that pass through a patient 112, and the data acquisition systems 122 converts the data to digital signals for subsequent processing. During a scan to acquire x-ray projection data, gantry 102 and the components mounted thereon, including the x-ray source 104 and the detector assembly 105, rotate about a center of rotation 114 so as to collect attenuation data from a multitude of view angles relative to the imaged volume.

As shown in FIG. 1, rotation of gantry 102 and the operation of x-ray source 104 are governed by a control mechanism 116 of CT system 100. Control mechanism 116 includes an x-ray controller 118 that provides power and timing signals to an x-ray source 104 and a gantry motor controller 120 that controls the rotational speed and position of gantry 102. An image reconstructor 124 receives sampled and digitized x-ray data from the data acquisition systems 122 and performs high-speed reconstruction. The reconstructed image is transmitted to a computer 126, which stores the image in a mass storage device 128. Computer 126 also receives commands and scanning parameters from an operator via console 130. An associated display 132 allows the operator to observe the reconstructed image and other data from computer 126. In addition, computer 126 operates a table motor controller 134, which controls a motorized table 136 to position patient 112 relative to the gantry 102. Particularly, table 136 moves (e.g., extends) portions of patient 112 on the patient support through a gantry opening or bore 138.

As noted above, to reduce motion artifacts, faster scanning speed is desired. However, existing CT scanners have the x-ray tube rotating together with the gantry. Thus, substantially increasing the gantry rotation speed is challenging because of the centripetal forces exerted on the components mounted on the gantry especially the fragile x-ray source. Currently the gantry rotation period of the fastest medical CT scanner (with single x-ray source) is close to 0.2 sec per revolution which still introduces motion artifacts, such as when imaging a human heart.

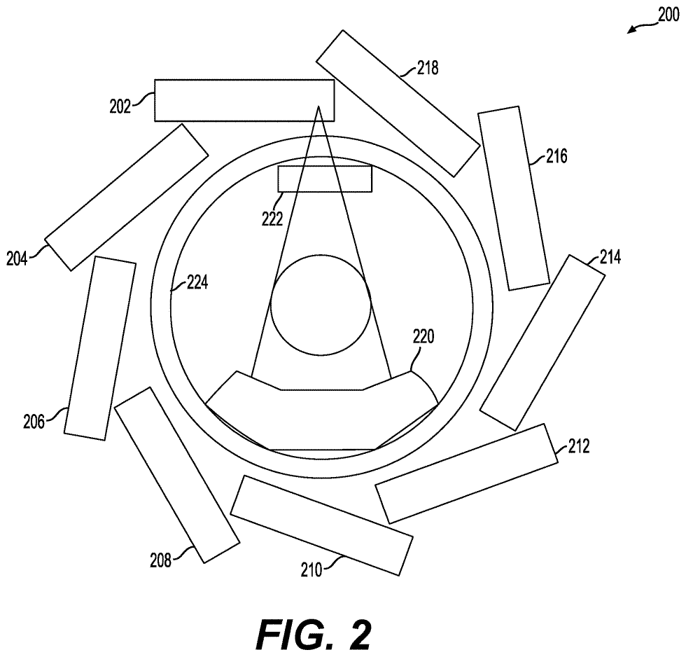

To further increase the scanning speed, a novel design of an x-ray tube is proposed. The newly proposed x-ray tube can generate x-rays continuously with x-ray foci forming a straight line, as discussed in more detail below. As shown in FIG. 2, a number of these x-ray tubes (202, 204, . . . , 218) and associated electronics are mounted outside a rotating gantry 224 of a CT scanner and kept stationary. In accordance with some embodiments, a filter 222 and a detector assembly 220 are mounted on gantry 224 and rotate together with gantry 224. In accordance with some embodiments, during scanning, x-rays emit from the stationary x-ray tubes (202, 204, . . . , 218) sequentially. At the same time, cone-angle collimator 222 and detector assembly 220 rotate together with gantry 224, such that the x-rays from an x-ray tube passes through cone-angle collimator 222 and reach detector assembly 220. In accordance with some embodiments, because the x-ray tubes are stationary, the gantry can rotate at a high speed of less than 0.1 sec per revolution to collect projection data. With this setup, patient motion can be effectively frozen so that the high-speed CT scanner can have a great impact on medical applications such as cardiac imaging problems. The high-speed CT scanner can also be operated at a relatively low speed to satisfy the routine applications of a CT scan.

### Exemplary X-Ray Tubes

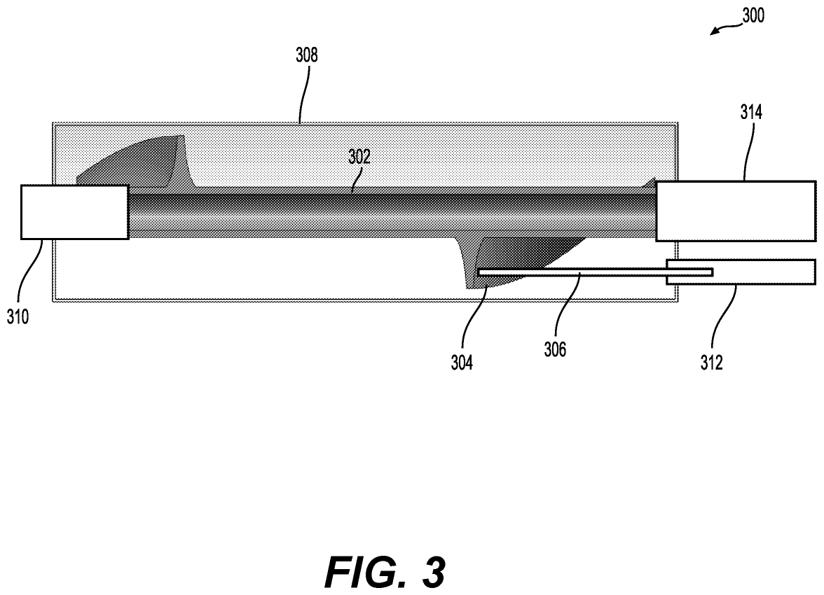

FIG. 3 is a schematic diagram of an x-ray tube in accordance with some embodiments. As shown, the x-ray tube 300 includes a rotatable member 302 having a rotation axis. A helical fin 304 is rigidly attached to the rotatable member 302, and the helical fin 304 is configured to rotate together with the rotatable member 302 around its rotation axis. The x-ray tube 300 also includes an electron beam generator 312 configured to generate at least one electron beam 306. The at least one electron beam 306 is directed to bombard the helical fin 304 as the helical fin rotates, producing x-rays whose focal spot moves in space along a line defined by the at least one electron beam 306. While the rotatable member 302 is illustrated as a cylinder in FIG. 3, other shapes, such as square column or cone, may be used.

As shown in FIG. 3, in accordance with some embodiments, the rotatable member 302 and the helical fin 304 are enclosed in a vacuum environment, which can be provided by a vacuumed glass cover 308. The bearing 310 and the motor 314 facilitate rotation of the rotatable member 302. The electron beam generator 312 and the vacuumed cover 308 may be enclosed in a housing (not shown). In accordance with some embodiments, the space between the vacuum cover 308 and the housing is filled with oil. To prevent possible space charge effects of the electron beam, external electric and/or magnetic fields will be applied outside or inside the vacuumed cover to keep the linear electron beam from spreading, so that the sizes of the x-ray focal spots on the helical fin are uniform. Such external fields can be formed in different ways. In accordance with some embodiments, the x-ray tube further includes a solenoid coil and/or a magnet inside or outside the vacuumed cover, the solenoid coil and/or the magnet being configured to generate a magnetic field to confine the electron beam. For example, thin coils made of Be or Al can be assembled around the vacuumed cover 308 to produce a magnetic field for confining the shape of the electron beam.

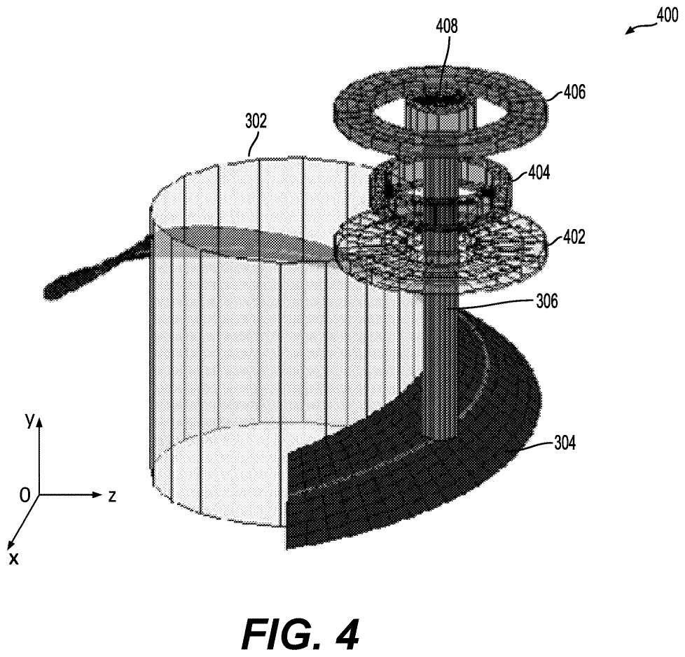

FIG. 4 is a schematic diagram illustrating one design of an x-ray tube in accordance with some embodiments. Similar to what is shown in FIG. 3, the x-ray tube 400 includes a rotatable member 302 and a helical fin 304 is rigidly attached to the rotatable member 302. The helical fin 304 is configured to rotate together with the rotatable member 302 around the rotation axis of the rotatable member 302 (e.g., parallel to y-axis). In some embodiments, the x-ray tube 400 may have a length of around 1000 mm and/or a diameter of about 200 mm. In some embodiments, the helical fin 304 may have a width of about 10 mm, a thickness of about 1 mm, and/or a slant angle of about 70 degree.

As shown in FIG. 4, the x-ray tube 400 further includes an electron beam generator, including one or more tungsten filaments 408, a control grid 406, an anode 402, and a focusing device 404. The tungsten filament 408 generates electrons when heated by electrical current. The control grid 406 can turn on/off the electron beam by changing the voltage on the grid. The anode 402 accelerates the electrons to a given energy/voltage. The focusing device 404 keeps the electron beam converging electrically or magnetically. In accordance with some embodiments, the electron beam generator is stationary (e.g., does not rotate with the rotatable member 302). At operation, a narrow beam of electrons 306 coming from the one or more tungsten filament 408 bombard on the surface of the helical fin 304 where x-rays will be produced. When the cylindrical part of the rotatable member 302 rotates around its axis (e.g., parallel to y-axis) together with the helical fin 304, the focal spots of the produced x-rays, which are the places where the electron beam hits the helical fin surface, will move along a straight line (e.g., parallel to y-axis). This is because the intersection points between the linear electron beam 306 and the surface of the helical fin 304 form a line parallel to the y-axis in space. In accordance with some embodiments, the helical fin 304 is made of or coated with tungsten or an alloy of tungsten and rhenium.

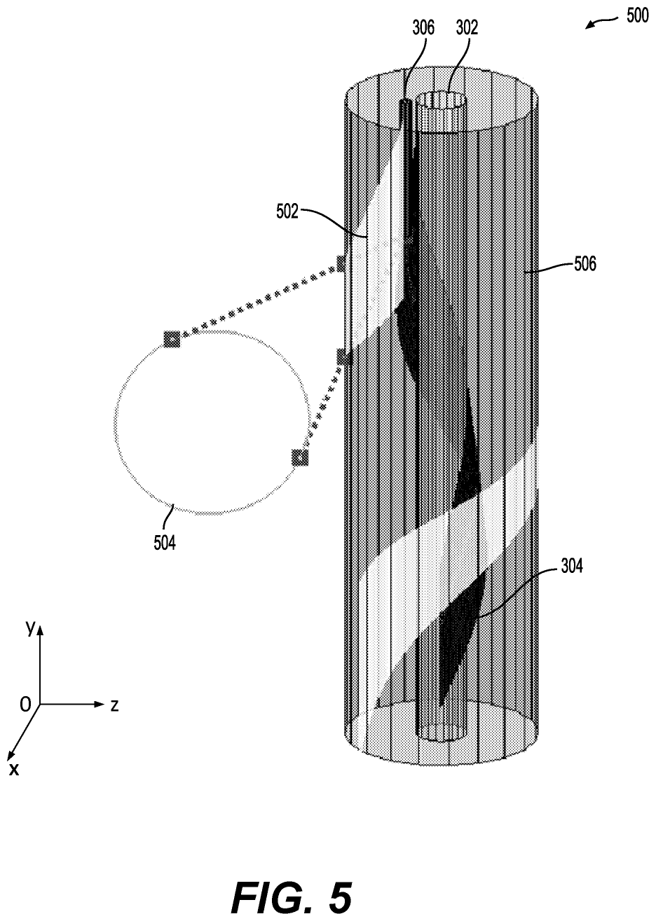

In accordance with some embodiments, the x-ray tube further includes a fan-angle collimator configured to collimate the produced x-rays by rotating in synchronization with the helical fin. As shown in FIG. 5, outside of the rotating member 302 and the rigidly attached helical fin 304 is covered by a fan-angle collimator 506. The fan-angle collimator 506 may be in a cylinder shape. In some embodiments, the fan-angle collimator 506 is arranged inside the vacuumed cover 308. The fan-angle collimator 506 connects the rotation axis of the helical fin (e.g., rotation axis of the rotatable member 302) and rotates in synchronization with the helical fin. A helical slotted band 502 is fabricated on the surface of the fan-angle collimator 506 and provides collimating effects. As shown in FIG. 5, the fan-angle collimator 506 includes a helical slotted band 502 having an upper boundary and a lower boundary, the upper boundary and the lower boundary being spiral curves, and the fan-angle collimator allows through a portion of the produced x-rays between the upper and lower boundaries of the helical slotted band 502. Outside the upper and lower boundaries of the helical slotted band 502, as illustrated in darker shade on the cylinder surface 506, x-rays are blocked by materials with high x-ray linear attenuation coefficient. In accordance with some embodiments, the fan-angle collimator 506 is configured to rotate with the rotatable member 302. In this way, at any given moment, x-rays emanating from a particular focal spot are confined within the two tangents of the central plane FOV circle 504.

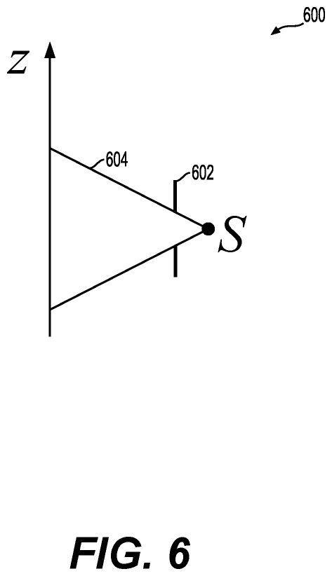

In accordance with some embodiments, the x-ray tube further includes a cone-angle collimator configured to collimate the portion of the produced x-rays allowed through by the fan-angle collimator. For example, as shown in FIG. 6, a cone-beam collimator 602 is designed to limit the cone-angle of the x-rays generated from source S. Here the cone-angle is defined as the angle subtended along segment direction (z-axis) by the x-rays from an x-ray source S sitting on the central plane. In some embodiments, the cone-beam collimator 602 is a simple rectangular slot which corresponds to a pre-defined cone-angle 604 along the z-axis for each x-ray source. Any x-rays that have cone-angle larger than a pre-defined one will be blocked by materials with high x-ray linear attenuation coefficients.

Exemplary High-Speed CT Scanners with Rotating Detectors

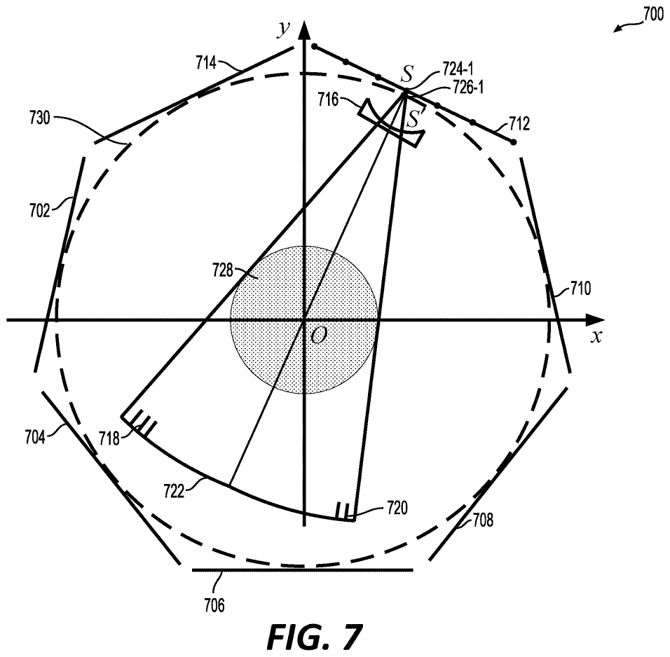

FIG. 7 is a schematic diagram illustrating a high-speed CT scanner setup including multiple x-ray tubes in accordance with some embodiments. As shown, a CT scanner 700 includes a plurality of x-ray tubes (702, 704, . . . , 714), each x-ray tube having a structure according to above description. The plurality of x-ray tubes (702, 704, . . . , 714) are stationary and form a closed polyline in 3D space around a field of view (FOV) 728 centering around an isocenter O. Because the x-ray tube can generate a set of linear x-ray sources, as described above, each x-ray tube is illustrated as a segment, as shown by segments 702, 704, . . . , 714.

In accordance with some embodiments, the number of the plurality of x-ray tubes depend on the length of each x-ray tube. In some embodiments, a number of the plurality of x-ray tubes is an odd number. For example, as shown in FIG. 7, the CT scanner 700 includes seven x-ray tubes. In some other embodiments, the number of the plurality of x-ray tubes is an even number.

Looking at one of the x-ray tubes, such as x-ray tube 712 in FIG. 7, the dots illustrated on x-ray tube 712, including 724-1, represent the focal spots of x-rays generated by x-ray tube 712. These plurality of x-ray tubes (702, 704, . . . , 714) are arranged to approximate a circle 730, representing the source trajectory (including S′) of a conventional CT scanner. S is referred to as a real focal spot of x-rays being generated by an x-ray tube. Since the x-ray source S′ on circle 730 is not present in the actual setup, S′ is referred to as a virtual source hereafter.

The CT scanner also includes a gantry (not shown) that is configured to rotate around the FOV 728. A first detector array 722 is rigidly attached to the gantry and is configured to rotate together with the gantry around the FOV 728 for collecting x-rays generated by the plurality of x-ray tubes (702, 704, . . . , 714). All these x-ray tubes and associated electronics such as high-voltage supplies are mounted outside of the rotating gantry and kept stationary. In some embodiments, the first detector array 722 is a curved detector array whose focus is at the virtual source position S′. In accordance with some embodiments, the first detector array includes anti-scatter grids (ASGs), such as ASGs 718 and 720. While FIG. 7 only illustrates the first detector array 722 as a curved detector, a cylindrical detector plane may be used to collect 2D projection data of a 3D object, and 1D or 2D ASGs are attached to the detector plane. In some embodiments, the CT scanner 700 also includes a bowtie filter mounted on the gantry and rotates with the gantry.

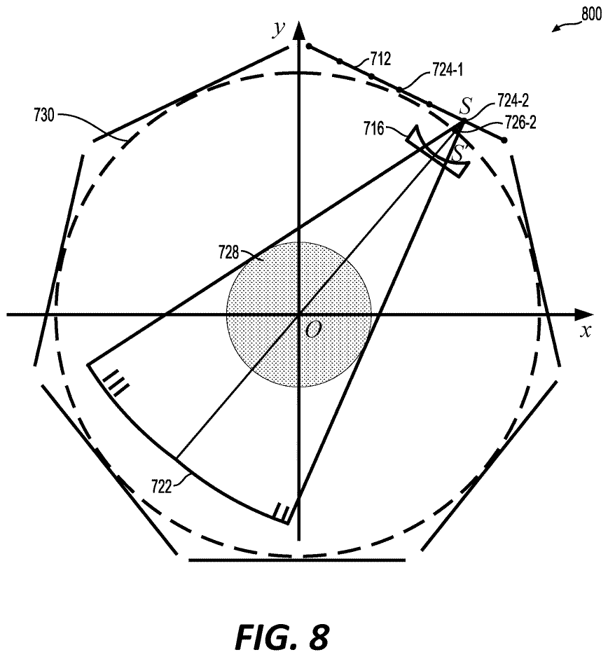

During operation, the first detector array 722 rotates with the gantry around the isocenter O. The rotation of the first detector array 722 and the rotation of a helical fin of a corresponding x-ray tube opposite to the first detector array 722 is synchronized such that a real focal spot (such as S) on the helical fin, a virtual source (such as S′) at a focus of the first detector array 722, and the isocenter O are colinear at every moment. For example, when the helical fin of x-ray tube 712 is rotated to a first rotation position, the real focal spot S is at location 724-1 of the x-ray tube 712. The corresponding virtual source S′ is at location 726-1 on circle 730, as shown in FIG. 7. At this time, the first detector array 722 is located at a first position such that the real focal spot S, the virtual source S′ at a focus of the first detector array 722, and the isocenter O are colinear. As the helical fin of x-ray tube 712 further rotates to a second rotation position, the real focal spot S moves from location 724-1 of the x-ray tube 712 to location 724-2, as shown in FIG. 8. Because the first detector array 722 rotates with the gantry around the isocenter O in synchronization with movement of the real focal spot S, when the real focal spot S moves to location 724-2, the corresponding virtual source S′ also moves accordingly to location 726-2 on circle 730, as shown in FIG. 8. At this moment, the real focal spot S, the virtual source S′ at a focus of the first detector array 722, and the isocenter O continues to be colinear. Same is true when the real focal spot moves from one x-ray tube to another.

In accordance with some embodiments, the closed polyline formed by the plurality of x-ray tubes (702, 704, . . . , 714) is a connected polyline. For example, as illustrated in FIG. 7, the plurality of x-ray tubes form the closed polyline within a first 2D plane (e.g., the x-o-y plane) in the 3D space. In some embodiments, the gantry is placed within a second 2D plane in the 3D space, and the second 2D plane is parallel to the first 2D plane (e.g., the x-o-y plane). In some embodiments, the second 2D plane is the same as the first 2D plane (i.e., the gantry and the plurality of x-ray tubes are placed in the same 2D plane).

In accordance with some other embodiments, the closed polyline formed by the plurality of x-ray tubes (702, 704, . . . , 714) is an unconnected polyline. For example, there is a slight spacing among the x-ray tubes forming the polygon in the z direction perpendicular to the x-o-y plane.

Since the x-tubes and associated electronics and power supplies are removed from the rotating gantry and kept stationary, the gantry can be rotated a lot faster than it is in the conventional CT scanner, resulting in a high-speed CT scanner. In some embodiments, the rotation time of the gantry can be less than 0.1 sec per revolution, which practically freezes motion of human hearts for example. To meet the routine CT application requirement, one needs only to adjust the rotation speed of the gantry and its synchronization procedure with the helical fin of the x-ray tubes to achieve a normal-speed operation mode. Once a complete set of projection data of an object is collected, analytic or iterative algorithms can be applied to reconstruct the image volumes.

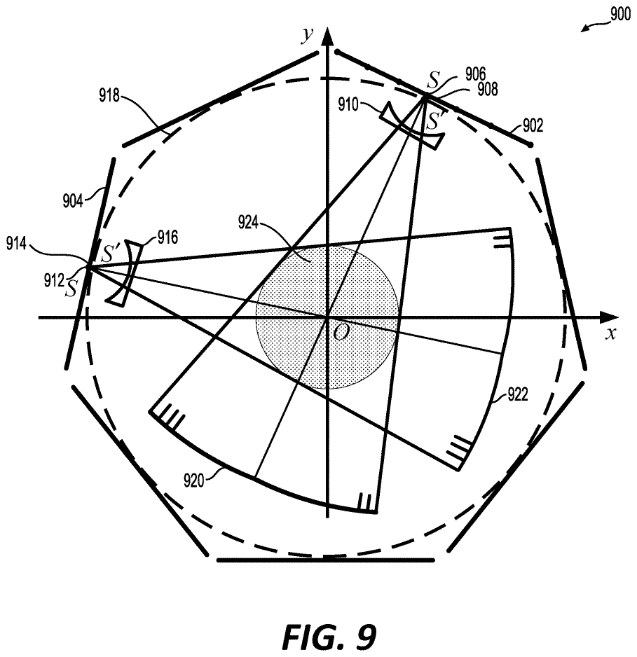

In accordance with some embodiments, a high-speed CT scanner further includes a second detector array, wherein the first and second detector arrays are configured to rotate together with the gantry around the FOV for collecting x-rays generated by the plurality of x-ray tubes. For example, as shown in FIG. 9, the CT scanner 900 includes a first detector array 920 and a second detector array 922. The first detector array 920 has a corresponding bowtie filter 910 and the second detector array 922 has a corresponding bowtie filter 916. As discussed above with respect to FIGS. 7 and 8, the rotation of the first detector array 920 and the rotation of a helical fin of a corresponding x-ray tube (such as x-ray tube 902) opposite to the second detector array 920 is synchronized such that a real focal spot S at location 906 on the helical fin of x-ray tube 902, the virtual source S′ at a focus of the first detector array 920 (e.g., location 908 on circle 918), and the isocenter O are colinear at every moment. Similarly, the rotation of the second detector array 922 and the rotation of a helical fin of a corresponding x-ray tube (such as x-ray tube 904) opposite to the second detector array 922 is synchronized such that a real focal spot S at location 912 on the helical fin of x-ray tube 904, the virtual source S′ at a focus of the second detector array 922 (e.g., location 912 on circle 918), and the isocenter O are colinear at every moment. In accordance with some embodiments, the two detector arrays 920 and 922 are separated by about 100 degree by a rotation angle. The two x-ray tubes opposite to the two detectors fire simultaneously and the data collecting time required is only half of the CT scanner with a single detector array.

Exemplary High-Speed CT Scanners with Stationary Detectors

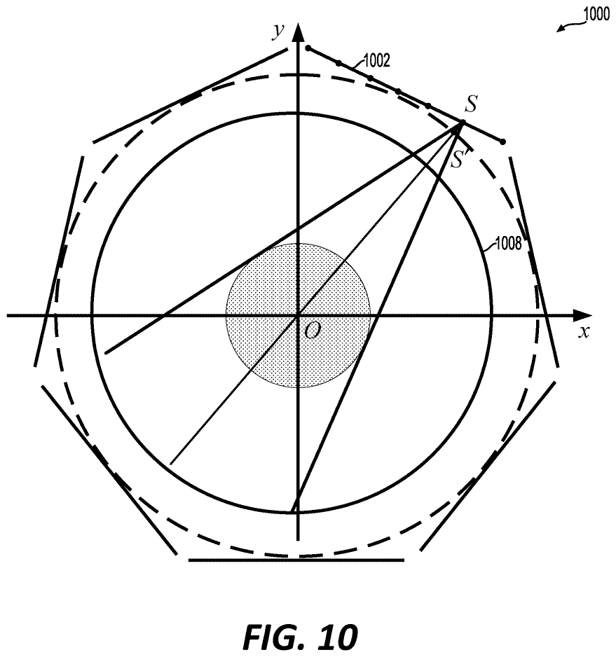

In addition to having one or more detector arrays that rotate together with the gantry, a high-speed CT scanner may also have one or more stationary detector arrays. As shown in FIG. 10, a CT scanner 1000 includes a plurality of x-ray tubes (such as x-ray tube 1002), each x-ray tube having a structure according to above description. The plurality of x-ray tubes are stationary and form a closed polyline in 3D space around a field of view (FOV). The CT scanner 1000 also includes a detector array 1008, which is stationary and surrounding the FOV and configured to collect x-rays generated by the plurality of x-ray tubes.

In accordance with some embodiments, the detector array 1008 includes detector elements arranged on a cylindrical face around the FOV. The plurality of x-ray tubes may fire sequentially for collecting the projection data. To reduce the data collecting time, multiple tubes may fire simultaneously as long as the detector array 1008 can differentiate the x-ray sources.

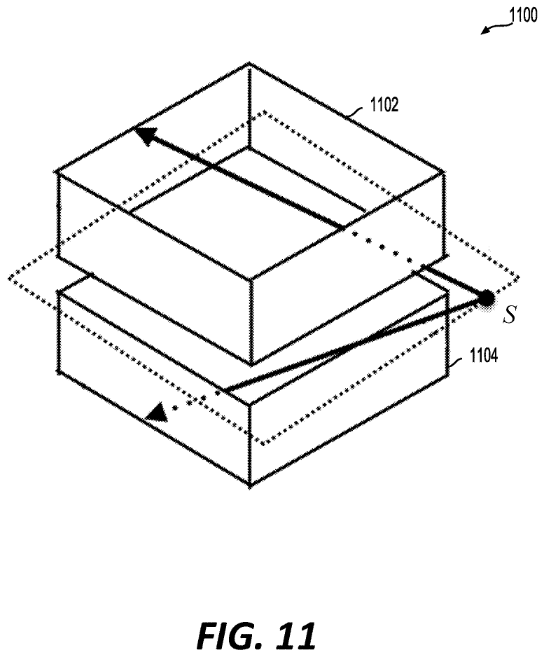

FIG. 11 is a schematic diagram illustrating a high-speed CT scanner setup with a stationary detector in accordance with some embodiments. For simplicity, the dotted segments represent the focal spots of x-rays generated by a plurality of x-ray tubes. While FIG. 11 only illustrates four dotted segments, it does not mean that there can exist only four stationary x-ray tubes. A person of ordinary skill in the art would have understood that the high-speed CT scanner setup 1100 can include any number of stationary x-ray tubes as described above. As shown in FIG. 11, focal spots of x-rays are arranged in the same 2D plane, indicating that the plurality of x-ray tubes are arranged in the same 2D plane.

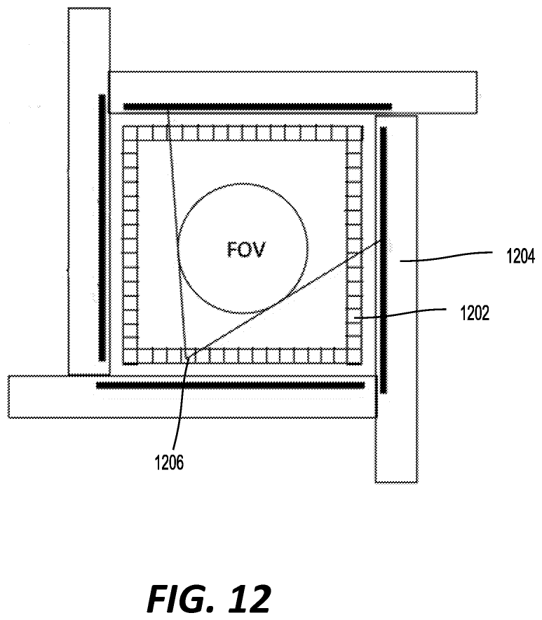

In accordance with some embodiments, the detector array is formed by a plurality of detector elements densely arranged in a 2D polyline face, as illustrated in FIG. 12. For example, as shown, detector elements 1202 are densely arranged in a 2D polyline face and configured to receive x-rays generated from x-ray tubes 1204. Specifically, detector element 1206 is configured to receive x-rays that come through within the FOV, as indicated by the fan shape in FIG. 12. By looking at a particular detector element with respect to the entire FOV (as opposed to looking at a particular x-ray source with respect to multiple detector elements), deviations (such as for responsiveness) between adjacent detector elements would not impact the reconstruction of the x-ray images.

If the 2D polyline face where the plurality of densely arranged detector elements locate overlap intersect with the 2D plane where the plurality of x-ray tube locate, the detector arrays may block x-rays generated by the plurality of x-ray tubes. In this situation, the detector elements are arranged offset the 2D plane where the x-ray tubes locate, so that the x-rays generated by each of the plurality of x-ray tubes can reach the FOV and not blocked by any detector element but at the same time ensuring that some detector elements are able to receive the projection data with sufficient energy. For example, as shown in FIG. 11, the detector array may include a gap within the 2D polyline face, the gap allowing the x-rays generated by at least one of the plurality of x-ray tubes to pass through. In order for the x-rays generated, for example at focal spot S, be transmitted unblocked and reach the detector elements of the stationary detector array, the detector array in the high-speed CT scanner setup 1100 includes a first detector array portion 1102 and a second detector array portion 1104 each having a plurality of detector elements. There is a gap between the first detector array portion 1102 and the second detector array portion 1104 in the direction perpendicular to the 2D plane where the plurality of x-ray tubes locate. Such a gap allows the x-rays generated, for example at focal spot S, pass through and reach the detector elements of the first detector array portion 1102 and/or the second detector array portion 1104.

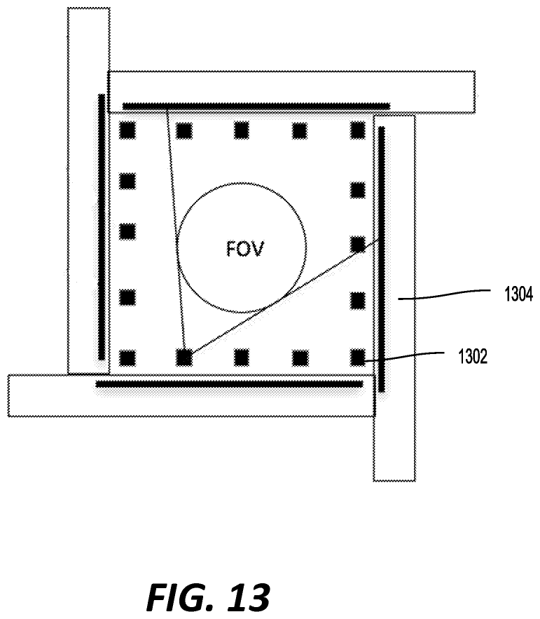

In accordance with some other embodiments, the detector array is formed by a plurality of detector elements sparsely arranged in a 2D polyline face, as illustrated in FIG. 13. For example, as shown, detector elements 1302 are sparsely arranged in a 2D polyline face and configured to receive x-rays generated from x-ray tubes 1304. Given that the detector elements are sparsely arranged and the size of each detector element is sufficiently small, the detector array may be able to capture sufficient x-rays generated at each focal spot without having a gap within the 2D polyline face as described in FIG. 11.

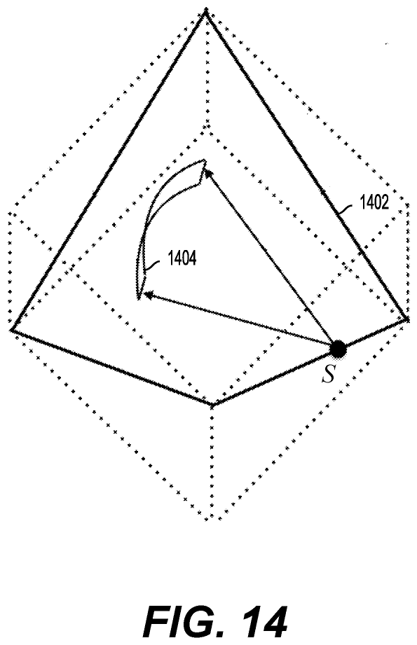

In accordance with some embodiments, the plurality of stationary x-ray tubes are not located in the same plane. For example, as shown in FIG. 14, the four solid lines indicate the x-ray source trajectory 1402 that is not in a plane. The dotted lines illustrate the frame of a cube. In this 4-tube case, any pair of two connected lines defines a plane, resulting in 4 planes in total. The volume enclosed by the 4 planes indicates an imaging space where exact reconstruction can be achieved. The projection data are capture by a detector array 1404, which may be stationary or not. This scan trajectory can eliminate the cone beam artifacts appearing in the 3D reconstruction from a circular scan or in general from scans with a trajectory in a plane since the data are not sufficient for 3D reconstruction. While FIG. 14 only illustrates four x-ray tubes. A person of ordinary skill in the art would have understood that the any number of x-ray tubes as described above may be used.

The high-speed CT scanners equipped with stationary x-ray tubes which produce x-rays with foci forming a straight line can have a great impact on medical applications, for example, cardiac imaging problems, where patient motion introduces difficulties. Such high-speed scanners can also be operated in relatively low-speed mode which satisfies the scenario of a routine CT scan. The stationary x-ray tubes which produce x-ray foci forming a straight line can find their applications in medical CT applications but also in industrial applications and homeland security inspection context and many more.

The following table summarizes qualitative comparisons of some technique specifications between the existing CT scanners and the proposed new high-speed CT (New-HSCT) scanner.

| | 3^{rd} Generation | Electron- | Stationary | New- |
|---|---|---|---|---|
| | CT | Beam CT | CT | HSCT |
| Is x-ray tube | Yes | No | No | No |
| rotating? | | | | |
| Is detector | Yes | No | No | Yes |
| rotating? | | | | |
| What is needed | Nothing | Magnetic | Nothing | Electric |
| to confine | | field | | and/or |
| the electron | | | | magnetic |
| beam? | | | | field |
| G-force on | Strong | No | No | No |
| x-ray source | | G-force | G-force | G-force |
| Can anti- | Yes | No | No | Yes |
| scatter grids | | | | |
| (ASG) be | | | | |
| installed? | | | | |
| Is it easy to | Yes | No | No | Yes |
| achieve large | | | | |
| z-coverage? | | | | |
| Power | Large | Small | Small | Large |
| Is it easy to | Yes | Yes | No | Yes |
| achieve high | | | | |
| density view | | | | |
| sampling? | | | | |
| Is it easy to | Yes | No | Yes | Yes |
| achieve small | | | | |
| focal spot? | | | | |
| Heat capacity | Large | Small | Small | Large |

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description, for purposes of explanation, has been described with reference to specific embodiments. However, the illustrative discussions above are not intended to be exhaustive or limit the claims to the precise forms disclosed. Many modifications and variations are possible in view of the above teachings. The embodiments were chosen and described in order to best explain principles of operation and practical applications, to thereby enable others skilled in the art.

What is claimed is:

1. An x-ray tube comprising:
a rotatable member having a rotation axis;
a helical fin rigidly attached to the rotatable member, wherein the helical fin is configured to rotate together with the rotatable member around the rotation axis; and
an electron beam generator configured to generate at least one electron beam,
wherein the at least one electron beam is directed to bombard the helical fin as the helical fin rotates, producing x-rays whose focal spot moves in space along a line defined by the at least one electron beam.

2. The x-ray tube according to claim, wherein the electron beam generator further includes one or more tungsten filaments, a control grid, an anode, and a focusing device.

3. The x-ray tube according to claim, wherein the helical fin is made of or coated with tungsten or an alloy of tungsten and rhenium.

4. The x-ray tube according to claim, wherein the x-ray tube further includes a fan-angle collimator configured to collimate the produced x-rays by rotating in synchronization with the helical fin.

5. The x-ray tube according to claim, wherein:
the fan-angle collimator includes a helical slotted band having an upper boundary and a lower boundary, the upper boundary and the lower boundary being spiral curves; and
the fan-angle collimator allows through a portion of the produced x-rays between the upper and lower boundaries of the helical slotted band.

6. The x-ray tube according to claim, wherein the fan-angle collimator is configured to rotate with the rotatable member.

7. The x-ray tube according to claim, wherein the x-ray tube further includes a cone-angle collimator configured to collimate the portion of the produced x-rays allowed through by the fan-angle collimator.

8. The x-ray tube according to claim, wherein the x-ray tube further includes a solenoid coil and/or a magnet inside or outside a vacuumed cover, the solenoid coil and/or the magnet being configured to generate a magnetic field to confine the electron beam.

9. A CT scanner, comprising:
a plurality of x-ray tubes, each x-ray tube having a structure according to claim 1, wherein the plurality of x-ray tubes are stationary and form a closed polyline in 3D space around a field of view (FOV) centering around an isocenter;
a gantry, wherein the gantry is configured to rotate around the FOV; and
a first detector array rigidly attached to the gantry, wherein the first detector array is configured to rotate together with the gantry around the FOV for collecting x-rays generated by the plurality of x-ray tubes,
wherein the rotation of the first detector array and the rotation of a helical fin of a corresponding x-ray tube opposite to the first detector array is synchronized such that a real focal spot on the helical fin, a virtual source at a focus of the first detector array, and the isocenter are colinear at every moment.

10. The CT-scanner according to claim, wherein a number of the plurality of x-ray tubes is an odd number.

11. The CT-scanner according to claim, wherein the closed polyline formed by the plurality of x-ray tubes is a connected polyline.

12. The CT-scanner according to claim, wherein the closed polyline formed by the plurality of x-ray tubes is an unconnected polyline.

13. The CT-scanner according to claim, further including a second detector array, wherein the first and second detector arrays are configured to rotate together with the gantry around the FOV for collecting x-rays generated by the plurality of x-ray tubes.

14. The CT-scanner according to claim, wherein a bowtie filter is mounted on the gantry and rotates with the gantry.

15. The CT-scanner according to claim, wherein the first detector array includes anti-scatter grids (ASGs).

16. The CT-scanner according to claim, wherein the plurality of x-ray tubes form the closed polyline within a first 2D plane in the 3D space.

17. The CT-scanner according to claim, wherein:
the gantry is placed within a second 2D plane in the 3D space; and
the second 2D plane is parallel to the first 2D plane.

18. The CT-scanner according to claim, wherein the second 2D plane is the same as the first 2D plane.

19. A CT scanner, comprising:
a plurality of x-ray tubes, each x-ray tube having a structure according to claim 1, wherein the plurality of x-ray tubes are stationary and form a closed polyline in 3D space around a field of view (FOV); and
a detector array, wherein the detector array is stationary and surrounding the FOV and configured to collect x-rays generated by the plurality of x-ray tubes.

20. The CT-scanner according to claim, wherein the detector array is formed by a plurality of detector elements densely arranged in a 2D polyline face.

21. The CT-scanner according to claim, wherein the detector array includes a gap within the 2D polyline face, the gap allowing the x-rays generated by at least one of the plurality of x-ray tubes to pass through.

22. The CT-scanner according to claim, wherein the detector array is formed by detector elements sparsely arranged in a 2D polyline face.
